(19) United States
(12) Patent Application Publication
(10) Pub. No.: US 2016/0340378 A1
(43) Pub. Date: Nov. 24, 2016

(54) METHOD FOR PURIFYING OXIDIZED FORM OF BETA-NICOTINAMIDE ADENINE DINUCLEOTIDE PHOSPHATE

(71) Applicant: BONTAC BIO-ENGINEERING (SHENZHEN) CO., LTD, Shenzhen (CN)

(72) Inventors: Rongzhao FU, SHENZHEN (CN); Zhu DAI, SHENZHEN (CN); Qi ZHANG, SHENZHEN (CN)

(21) Appl. No.: 15/109,964

(22) PCT Filed: Dec. 4, 2015
(86) PCT No.: PCT/CN2015/096408

§ 371 Date: Jul. 6, 2016

(30) Foreign Application Priority Data
May 19, 2015 (CN) 201510255144.6

(51) Int. Cl. C07H 1/06; C07H 19/207

(52) U.S. Cl. CPC C07H 1/06; C07H 19/207

(57) ABSTRACT

The present invention discloses an oxidized form of β-nicotinamide adenine dinucleotide phosphate and a method for purifying the same. The method comprises specifically the steps of: a. sequentially microfiltrating and nanofiltrating a pretreated coenzyme II solution with membrane concentration devices, to obtain a concentrated crude product solution; b. adjusting the obtained crude product solution to pH 2-4, loading it onto a preparative reverse phase high performance liquid chromatographic column, and purifying by gradient elution, to obtain a purified sample solution; and c. nanofiltrating the purified sample solution with a membrane concentration device, and freeze drying it in a vacuum freeze drier, to obtain a purified coenzyme II. The coenzyme II prepared in the present invention has a high purity, a high yield, and thus a promising prospect in the market.

### BACKGROUND

1. Technical Field

The present invention relates to the field of nucleotide coenzymes, and particularly to a method for purifying oxidized form of β-nicotinamide adenine dinucleotide phosphate.

2. Related Art

Oxidized form of β-nicotinamide adenine dinucleotide phosphate (NADP, also known as coenzyme II) is an important nucleotide coenzyme, which is a derivative of nicotinamide adenine dinucleotide (NAD) obtained through phosphorylation of position 2′ of the ribose ring system attached to adenine, and involved in a variety of anabolic reactions, including the synthesis of lipids, fatty acid, and nucleotides. The coenzyme II functions in organisms not only as a carrier for hydrogen transfer, but also as a medium for phosphoryl transfer to participate in various synthesis reactions.

The coenzyme II is widely used, and currently purified by conventional processes including ion exchange and other means. However, due to the influence arising from the process for synthesizing the coenzyme II, a large amount of phosphate is generally contained in the crude product. The phosphate is difficult to be removed by ion exchange, and cannot be completely removed during concentration by nanofiltration. Therefore, the problem of phosphate residue in the product is difficult to be solved. Moreover, the purity of the coenzyme II obtained by ion exchange is only about 95%, and the yield is only 60%, such that the production capability is greatly limited, and cannot meet the demand in the market. Accordingly, the domestic demand can only be met by importation, which increases the enterprise and industry cost.

Therefore, improvements and developments are needed in the art.

### SUMMARY

### Technical Problem

In view of the defects existing in the prior art, an object of the present invention is to provide a method for purifying oxidized form of β-nicotinamide adenine dinucleotide phosphate, for the purpose of addressing the problems of difficulty in removal of phosphate, and low purity and low yield of the product existing in the prior art.

### Technical Solution

To achieve the above object, the following technical solution is adopted in the present invention.

A method for purifying oxidized form of β-nicotinamide adenine dinucleotide phosphate comprises the steps of:

a. sequentially microfiltrating and nanofiltrating a pretreated coenzyme II solution with membrane concentration devices, to collect a concentrated crude product solution;

b. adjusting the obtained crude product solution to pH 2-4, loading it onto a preparative reverse phase high performance liquid chromatographic column, and purifying by gradient elution using a phenyl bonded silica gel as a stationary phase, a hydrochloric acid solution at pH 2-4 as a mobile phase A, and ethanol as a mobile phase B, to obtain a purified sample solution; and

c. nanofiltrating the purified sample solution with a membrane concentration device, and freeze drying it in a vacuum freeze drier, to obtain a purified coenzyme II.

In the method for purifying oxidized form of β-nicotinamide adenine dinucleotide phosphate, the microfiltration membrane used for microfiltration in Step a has a pore size of 0.2-1 μm.

In the method for purifying oxidized form of β-nicotinamide adenine dinucleotide phosphate, the nanofiltration membrane used for nanofiltration in Step a has a 200 molecular weight cut-off.

In the method for purifying oxidized form of β-nicotinamide adenine dinucleotide phosphate, the nanofiltration membrane used for nanofiltration in Step a is a hollow fiber membrane.

In the method for purifying oxidized form of β-nicotinamide adenine dinucleotide phosphate, the concentration of the crude product solution concentrated in Step a is 20-30 g/L.

In the method for purifying oxidized form of β-nicotinamide adenine dinucleotide phosphate, the concentration of the purified sample solution after nanofiltration with a membrane concentration device in Step c is 100-150 g/L.

In the method for purifying oxidized form of β-nicotinamide adenine dinucleotide phosphate, the nanofiltration membrane used for nanofiltration in Step c is a hollow fiber membrane with a 200 molecular weight cut-off

In the method for purifying oxidized form of β-nicotinamide adenine dinucleotide phosphate, the volume ratio of the mobile phase A to the mobile phase B in Step b is from 1:99 to 1:1.

In the method for purifying oxidized form of β-nicotinamide adenine dinucleotide phosphate, the elution time for gradient elution in Step b is 40 min.

### Beneficial Effect

By purifying the coenzyme II by preparative reverse phase high performance liquid chromatography on phenyl bonded silica gel in the present invention, the purity of the resulting product is up to 99%, the yield is 90% or more, and the production efficiency is increased by more than 80% compared with other processes, thus greatly reducing the production cost, and effectively solving the problems existing in the prior art of difficulty in removal of phosphate residue, and low purity and low yield of the prepared product.

### DETAILED DESCRIPTION

The present invention provides a method for purifying oxidized form of β-nicotinamide adenine dinucleotide phosphate. To make the objects, technical solutions, and effects of the present invention clearer and more precise, the present invention is described in further detail hereinafter. It should be understood that the specific embodiments described herein are merely provided for illustrating, instead of limiting the present invention.

A method for purifying oxidized form of β-nicotinamide adenine dinucleotide phosphate comprises the steps of:

S101: sequentially microfiltrating and nanofiltrating a pretreated coenzyme II solution with membrane concentration devices, to collect a concentrated crude product solution;

S102: adjusting the obtained crude product solution to pH 2-4, loading it onto a preparative reverse phase high performance liquid chromatographic column, and purifying by gradient elution using a phenyl bonded silica gel as a stationary phase, a hydrochloric acid solution at pH 2-4 as a mobile phase A, and an ethanol solution as a mobile phase B, to obtain a purified sample solution; and

S103: nanofiltrating the purified sample solution with a membrane concentration device, and freeze drying it in a vacuum freeze drier, to obtain a purified coenzyme II.

In an embodiment, the coenzyme II is purified by reverse phase high performance liquid chromatography. Specifically, a coenzyme II solution is initially concentrated, then purified by column chromatography using a phenyl bonded silica gel as a stationary phase, and a hydrochloric acid solution and an ethanol solution as mobile phases, and further concentrated and freeze dried, to obtain a purified coenzyme II. By purifying the coenzyme II by reverse phase high performance liquid chromatography on phenyl bonded silica gel in the present invention, the resulting coenzyme II has a high purity, a high yield, and a large output, and the problem existing in the prior art of difficulty in removal of phosphate residue can be effectively solved. The process of the present invention is simple, and applicable to the large scale production and purification of coenzyme II in industry.

Preferably, the microfiltration membrane used for microfiltration in Step S101 has a pore size of 0.2-1 μm. For example, in a preferred embodiment of the present invention, the microfiltration membrane used for microfiltration in Step S101 has a pore size of 0.5 μm.

The fundamental principle underlying microfiltration is sieving. Particles with a particle size of 10 μm or greater are filtered off under a differential static pressure. Under an operation pressure of 0.7-7 bar, the solvent in the raw solution penetrates through the micropores on the membrane and flows to a low pressure side of the membrane under the differential pressure, and the particles with a particle size larger than the pore size of the membrane is retained, thereby realizing the separation of particles from the solvent in the raw solution. The mechanism of retaining the particles by microfiltration is sieving. The separation effect of the membrane depends on the physical structure and the pore shape and size of the membrane.

Where the microfiltration membrane used for microfiltration in an embodiment has a pore size of 0.5 μm, large microorganisms and particles in the crude coenzyme II product solution can be preliminarily removed. The microfiltration membrane allows macromolecules and dissolved solid (inorganic) salts to pass through, but retains suspended matter, bacteria, and high molecular weight colloids, thereby achieving the preliminary purification for the crude coenzyme II product solution. If the pore size of the microfiltration membrane is too large, large microorganisms and particles may penetrate through the microfiltration membrane, thus affecting the effect of preliminary filtration. If the pore size is too small, the coenzyme II may be caused to fail to penetrate through the microfiltration membrane, leading to the loss of the product.

Preferably, the nanofiltration membrane used for nanofiltration in Step S101 in an embodiment of the present invention has a 200 molecular weight cut-off.

More preferably, the nanofiltration membrane used for nanofiltration in Step S101 is a hollow fiber membrane.

Nanofiltration is a filtration method that permits the solvent molecules, some small molecular weight solutes, or low valence ions to penetrate through, and is characterized by having a high salt removal performance and capability to retain materials with a molecular weight of several hundreds under a quite low pressure, and having a high retention rate for di- or higher valent ions and particularly anions. In the present invention, a nanofiltration membrane with a 200 molecular weight cut-off is used, whereby the materials with a molecular weight of 200 or above are filtered off. Moreover, by using a hollow fiber membrane as the nanofiltration membrane, the phosphate residue and other small molecular weight impurities produced during the process for preparing the coenzyme II can be effectively removed, such that the coenzyme II is further purified.

In a preferred embodiment of the present invention, the concentration of the phosphoric acid solution or the hydrochloric acid solution in Step S102 is 2-5 wt %, and the concentration of the ethanol solution is 5-30 wt %.

More preferably, the phosphoric acid solution or the hydrochloric acid solution in Step S102 is used in an amount (by volume) of 5-20 ml of the phosphoric acid solution or the hydrochloric acid solution per ml of the sample solution, and the ethanol solution is used in an amount (by volume) of 100-400 ml of the ethanol solution per L of the mobile phases.

Specifically, if the mass concentration of the phosphoric acid solution or the hydrochloric acid solution is too high, eduction is caused during the gradient elution; and if the mass concentration is too low, the separation and purification effect is affected. In the present invention, high degree of purification of the coenzyme II can be achieved with the use of 2-5 wt % phosphoric acid solution or hydrochloric acid solution without eduction during the gradient elution.

Moreover, varying amount of acid may have an impact on the peak shape, thus affecting the detection effect. In an embodiment of the present invention, the phosphoric acid solution or the hydrochloric acid solution is used in an amount (by volume) of 5-20 ml of the phosphoric acid solution or the hydrochloric acid solution per ml of the sample solution, and the ethanol solution is used in an amount (by volume) of 100-400 ml of the ethanol solution per L of the mobile phases, such that the peak shape is symmetric and the tailing is reduced.

In Step S102 in the present invention, the obtained crude product solution is adjusted to pH 2-4 with a phosphoric acid solution or a hydrochloric acid solution, because the crude product solution of coenzyme II still contains a phosphate residue; and a solution formulated with hydrochloric acid is used as the mobile phase A. In the present invention, no new impurities are introduced by adjusting the pH value of the crude product solution with a phosphoric acid solution or a hydrochloric acid solution, and the phosphate and others can be removed in the purification process.

Specifically, during the reverse phase high performance liquid chromatography, generally a non-polar stationary phase (e.g. C18, or C8) is used, and the mobile phase is water or a buffer, to which methanol, ethanol, isopropanol, acetone, tetrahydrofuran and other organic solvents that are miscible with water are usually added to adjust the retention time. These are suitable for the separation of non-polar or weakly polar compounds. The pH affects the existing state of the sample, and thus affects the retention time. In an embodiment of the present invention, a hydrochloric acid solution at pH 2-4 and ethanol are used as the mobile phases, such that the retention time of the sample is effectively adjusted, and the time from the entering of the sample into the chromatographic column to existing the chromatographic column is optimized, thereby leading to a good separation effect for the sample. If the retention time of the sample is too long, the detection sensitivity is lowered; and if the retention time of the sample is too short, the effect of separating the coenzyme II from the impurities is decreased, thus influencing the purification of the coenzyme II.

Preferably, the volume ratio of the mobile phase A to the mobile phase B in Step S102 is 1:99-1:1. For example, in a preferred embodiment of the present invention, the volume ratio of the mobile phase A to the mobile phase B is 50:100.

The ratio of the mobile phases has an influence on the detection and purification effects of the sample. If the amount of the hydrochloric acid solution is increased and the amount of ethanol is decreased, the retention time of the sample is extended, and the sample is well separated. However, the sample peak detected is widened, and the peak height is reduced, thus affecting the detection sensitivity. If the amount of the hydrochloric acid solution is decreased and the amount of ethanol is increased, the detection sensitivity of the sample is high, but the separation effect of the sample is poor. In the present invention, the hydrochloric acid solution at pH 2-4 and ethanol are used at a volume ratio of 1:99-1:1, such that the detection sensitivity of the sample is increased while the sample is well separated and purified.

Preferably, where the elution time in gradient elution in Step S102 is 40 min, the prepared coenzyme II may have a high purity, and 90% or more of the coenzyme II can be eluted off, thereby improving the production.

In Step S103, the purified sample solution is nanofiltrated with a membrane concentration device, and then freeze dried in a vacuum freeze drier, to obtain a coenzyme II with a purity of 99% and a yield of 90% or above.

In the present invention, compared with isocratic elution, the use of gradient elution can provide a peak that is more symmetric and has no tailing, and can improve the column efficiency and the detection sensitivity. Furthermore, an elution time of 40 min allows the sample to have a suitable retention time, so that a most desirable separation and purification may be achieved for the coenzyme II. In particular, the purification is carried out by eluting with a gradient of 1% to 10% B.

The present invention is further explained with reference to specific examples.

Chromatographic columns with a specification (column diameter *length): 5 cm*30cm, 15 cm*30 cm, and 30 cm*30 cm are used.

The column temperature is room temperature.

### EXAMPLE I

1. Concentration of crude product:

A pretreated coenzyme II solution was microfiltrated and nanofiltrated with membrane concentration devices, where the microfiltration was carried out for removing microorganisms, and a hollow fiber membrane with a 200 molecular weight cut-off was used for nanofiltration. As a result, the crude product was concentrated to 40-60 g/L.

2. Purification:

Purification conditions:

Chromatographic column: 5 cm*30 cm;

Stationary phase: phenyl bonded silica gel;

Mobile phases: Phase A: hydrochloric acid solution at pH 2; and Phase B: ethanol.

Flow rate: 50-80 ml/min;

Detection wavelength: 260 nm;

Gradient: B%: 1%-10% (over 40 min); and

Amount of injection: 10-15 g.

Purification process: The concentrated crude product solution was adjusted to pH 2-4 with a phosphoric acid solution or a hydrochloric acid solution. The chromatographic column was rinsed with 30% or above of ethanol, equilibrated, and injected with the sample in an amount of 10-15 g. The sample was eluted for 40 min with a linear gradient, and the target peak was collected.

Concentration and freeze drying:

The purified sample solution was concentrated to 100-150 g/L by nanofiltrating using a membrane concentration device (a hollow fiber membrane with a 200 molecular weight cut-off), and then freeze dried in a vacuum freeze drier, to obtain a freeze dried coenzyme II product with a purity that is higher than 99% and a total yield that can be up to 90.8%.

### EXAMPLE II

1. Concentration of crude product:

A pretreated coenzyme II solution was microfiltrated and nanofiltrated with membrane concentration devices, where the microfiltration was carried out for removing microorganisms, and a hollow fiber membrane with a 200 molecular weight cut-off was used for nanofiltration. As a result, the crude product was concentrated to 40-60 g/L.

2. Purification:

Purification conditions:

Chromatographic column: 15 cm*30 cm;

Stationary phase: phenyl bonded silica gel;

Mobile phases: Phase A: hydrochloric acid solution at pH 3; and Phase B: ethanol;

Flow rate: 400-500 ml/min;

Detection wavelength: 260 nm;

Gradient: B%: 1%-10% (over 40 min); and

Amount of injection: 70-90 g.

Purification process: The concentrated crude product solution was adjusted to pH 2-4 with a phosphoric acid solution or a hydrochloric acid solution. The chromatographic column was rinsed with 30% or above of ethanol, equilibrated, and injected with the sample in an amount of 70-90 g. The sample was eluted for 40 min with a linear gradient, and the target peak was collected.

3. Concentration and freeze drying: The purified sample solution was concentrated to 100-150 g/L by nanofiltrating using a membrane concentration device (a hollow fiber membrane with a 200 molecular weight cut-off), and then freeze dried in a vacuum freeze drier, to obtain a freeze dried product with a purity that is higher than 99% and a total yield that can be up to 91.5%.

### EXAMPLE III

1. Concentration of crude product:

A pretreated coenzyme II solution was microfiltrated and nanofiltrated with membrane concentration devices, where the microfiltration was carried out for removing microorganisms, and a hollow fiber membrane with a 200 molecular weight cut-off was used for nanofiltration. As a result, the crude product was concentrated to 40-60 g/L.

2. Purification:

Purification conditions:

Chromatographic column: 30 cm*30cm;

Stationary phase: phenyl bonded silica gel;

Mobile phases: Phase A: hydrochloric acid solution at pH 4; and Phase B: ethanol;

Flow rate: 2500-3000 ml/min;

Detection wavelength: 260 nm;

Gradient: B%: 1%-10% (over 40 min); and

Amount of injection: 400-500 g.

Purification process: The concentrated crude product solution was adjusted to pH 2-4 with a phosphoric acid solution or a hydrochloric acid solution. The chromatographic column was rinsed with 30% or above of ethanol, equilibrated, and injected with the sample in an amount of 400-500 g. The sample was eluted for 40 min with a linear gradient, and the target peak was collected.

3. Concentration and freeze drying:

The purified sample solution was concentrated to 100-150 g/L by nanofiltrating using a membrane concentration device (a hollow fiber membrane with a 200 molecular weight cut-off), and then freeze dried in a vacuum freeze drier, to obtain a freeze dried product with a purity that is higher than 99% and a total yield that can be up to 91.4%.

In the present invention, a target peak of the sample is collected, and nanofiltrated with a membrane concentration device when a standard is met, followed by freeze drying in a vacuum freeze drier, to obtain the coenzyme II prepared and purified in the present invention. The freeze dried coenzyme II product prepared following the method of the present invention has a purity that is up to 99% and total yield that can be up to 90% or above. The method of the present invention is simple in operation, and the production efficiency is increased by more than 80% compared with other processes, thereby effectively solving the problems existing in the prior art of difficulty in removal of phosphate residue, and low purity and low yield of the prepared product.

It should be understood that the present invention is not limited to the embodiments above, and equivalent replacements or changes may be made by those ordinarily skilled in the art based on the description, which are all embraced in the protection scope as defined by the accompanying claims of the present invention.

What is claimed is:

1. A method for purifying oxidized form of β-nicotinamide adenine dinucleotide phosphate, comprising the steps of:
a. sequentially microfiltrating and nanofiltrating a pretreated coenzyme II solution with membrane concentration devices, to collect a concentrated crude product solution;
b. adjusting the obtained crude product solution to pH 2-4, loading it onto a preparative reverse phase high performance liquid chromatographic column, and purifying by gradient elution using a phenyl bonded silica gel as a stationary phase, a hydrochloric acid solution at pH 2-4 as a mobile phase A, and ethanol as a mobile phase B, to obtain a purified sample solution; and
c. nanofiltrating the purified sample solution with a membrane concentration device, and freeze drying it in a vacuum freeze drier, to obtain a purified coenzyme II.

2. The method for purifying oxidized form of β-nicotinamide adenine dinucleotide phosphate according to claim 1, wherein the microfiltration membrane used for microfiltration in Step a has a pore size of 0.2-1 μm.

3. The method for purifying oxidized form of β-nicotinamide adenine dinucleotide phosphate according to claim 1, wherein the nanofiltration membrane used for nanofiltration in Step a has a 200 molecular weight cut-off

4. The method for purifying oxidized form of β-nicotinamide adenine dinucleotide phosphate according to claim 1, wherein the nanofiltration membrane used for nanofiltration in Step a is a hollow fiber membrane.

5. The method for purifying oxidized form of β-nicotinamide adenine dinucleotide phosphate according to claim 1, wherein the concentration of the crude product solution concentrated in Step a is 20-30 g/L.

6. The method for purifying oxidized form of β-nicotinamide adenine dinucleotide phosphate according to claim 1, wherein the concentration of the purified sample solution after nanofiltration with a membrane concentration device in Step c is 100-150 g/L.

7. The method for purifying oxidized form of β-nicotinamide adenine dinucleotide phosphate according to claim 1, wherein the nanofiltration membrane used for nanofiltration in Step c is a hollow fiber membrane with a 200 molecular weight cut-off

8. The method for purifying oxidized form of β-nicotinamide adenine dinucleotide phosphate according to claim 1, wherein the volume ratio of the mobile phase A to the mobile phase B in Step b is from 1:99 to 1:1.

9. The method for purifying oxidized form of β-nicotinamide adenine dinucleotide phosphate according to claim 1, wherein the elution time for gradient elution in Step b is 40 min.
